(19) United States
(12) Patent Application Publication
(10) Pub. No.: US 2024/0058325 A1
(43) Pub. Date: Feb. 22, 2024

(54) IMPROVED INDUSTRIAL PROCESS FOR EXTRACTION OF ALPHA YOHIMBINE FROM RAUWOLFIA SPECIES AND THE EXTRACT THEREOF

(71) Applicant: GOEL PAWAN KUMAR, Panchkula (IN)

(72) Inventors: MANOJ BALI, Panchkula (IN); ASHOK KUMAR CHAUHAN, Panchkula (IN); KIRAN TIWARI, Panchkula (IN)

(73) Assignee: GOEL PAWAN KUMAR, Panchkula (IN)

(21) Appl. No.: 17/766,410

(22) PCT Filed: May 5, 2021
(86) PCT No.: PCT/IN2021/050506

§ 371 Date: Apr. 4, 2022

(30) Foreign Application Priority Data
Mar. 23, 2021 (IN) 202111012485

(51) Int. Cl. A61K 31/475; A61K 36/24

(52) U.S. Cl. CPC A61K 31/475; A61K 36/24; A61K 2236/15; A61K 2236/33; A61K 2236/53; A61K 2236/51; A61K 2236/35

(57) ABSTRACT

The present invention discloses an improved process for the extraction of alpha yohimbine from leaves of Rauwolfia species, preferably Rauwolfia canescens/Rauwolfia tetraphylla. Process involves use of non-polar alcoholic solvent e.g. methanol for precipitating the compound of interest, repetitive washing with halogenated solvents and washing with hexane or petroleum ether to obtain high yields of high purity extract. Use of organic acids such as tartaric acid, acetic acid and organic solvent ethyl acetate etc. is eliminated making the process less time consuming and economical. The yield of the final product is 50-60% higher i.e. 600-670 mg/100 g dry leaves, than prior art process. Further, the extract obtained by the process of present invention contains yohimbine hydrochloride as a marker compound in analytically detectable amounts of more than 0.1% of the extract.

### FIELD OF INVENTION

The invention generally relates to Plant Resources. More specifically, it pertains to an improved industrial process for the extraction of Alpha yohimbine from Rauwolfia species and the extract thereof. The process produces alpha yohimbine with a higher yield in a faster and more economical manner, as compared to prior art processes.

### BACKGROUND OF THE INVENTION

### Rauwolfia and its Importance:

Rauwolfia is a genus of Evergreen trees and shrubs belonging to dogbane or Apocynaceae family. Rauwolfia genus comprises of about 100 species. Plants belonging to genus of Rauwolfia are native to tropical or sub-tropical regions of world including Europe, Africa, Asia, Australia, and the Central and South America. Rauwolfia typically grows at a temperature of about 10-40° C. Rauwolfia species are of great medicinal and economic importance because of the presence of different Phytochemicals including alcohols, sugars and glycosides, Flavonoids, Steroids, tannins and Alkaloids.

All parts of Rauwolfia plant including, roots, stems and leaves comprises of Indole Alkaloids. The major Indole Alkaloids present in Rauwolfia are ajmalidine, ajmaline, aricine, canescine, isoserine, serpentine. Serpentitnine, reserpiline, reserpine, Yohimbine, Yohimbinine etc.

https://www.ncbi.nlm.nih.gov/pmc/articles/PMC4566472/).

In India, about 8 species of genus Rauwolfia are cultivated. Rauwolfia serpentina is also known as Indian Snakeroot or Sarpagandha. It consists of various alkaloids and is most commonly used in the field of medicine. It is used for the treatment of hypertension, psychotic disorders, intestinal disorders, etc. It is also used to treat Poisonous insect stings and snake bites. Rauwolfia canescens/Rauwolfia tetraphylla also known as devil-pepper, also contain numerous phytochemicals and is of great medicinal importance. Different species of Rauwolfia contain different amounts of phytochemicals.

Alpha Yohimbine—Yohimbine is one of the major Indole/carboline alkaloid, whose major source is the bark of Pausinystalia species preferably Pausinystalia yohimbe (Corynanthe yohimbe). A remarkable amount of yohimbine is also present in Rauwolfia species, Pseudochinchona species etc.

The spatial arrangement of functional group (—OH) and properties of alpha yohimbine are different from those of yohimbine. Alpha Yohimbine is one of the diastereoisomers of alkaloid yohimbine.

### Prior Art Methods of Extraction of Alpha Yohimbine

Various methods are present for the extraction of alpha yohimbine from Rauwolfia species, such as:
1. Solvent based methods using organic solvent alkalization extraction and organic acid acidic extraction—utilizes non polar aromatic solvent “toluene” for extraction along with use of various organic acids such as tartaric acid, oxalic acid etc. This type of extraction is used at Industrial Scale.
2. Column Chromatographic method using organic solvent, alkali and acidic extraction step for extraction—Mainly used at R&D or lab scale.
3. High Performance Thin Layer Liquid Chromatography—Extraction of compound and purification and quantitative determination of compound is also carried out using HPTLC. Mainly used at R&D or lab scale.

### Drawbacks of Prior Art Process

Most of prior art methods involve the use of sophisticated techniques or equipment such as Column Chromatography. Reverse phase chromatography, HPLC coupled with ion trap mass spectrometry etc. which are quite complex, time consuming and not economical on industrial scale.

Further the prior art methods are resulting in low yield of the product despite use of expensive solvents and a number of steps.

Also the previous process involves the use of a number of expensive solvents/reagents such as oxalic acid, tartaric acid, ethyl acetate etc.

### Innovative Approach Used by the Inventors

Present invention overcomes these drawbacks and offers technical and economic advantages not offered by any of the prior art processes.

Process of present invention gives much higher yield than prior art processes, due to use of less number of solvents/reagents and process steps.

Use of less number of process steps help to reduce handling losses as suffered in previous processes, thus increasing yield.

Process of present invention is much more economical due to following reasons:—
i. Does not involve use of sophisticated and complex chromatography and spectroscopy techniques
ii. Uses less number of reagents/solvents which decreases losses and reduces cost
iii. Uses cheaper reagents/chemicals/solvents
iv. Uses lesser number of steps thus saving man power and time

### PRIOR ART PATENTS

U.S. Pat. No. 10,730,869 B2 discloses “Novel Process for Extracting Alpha Yohimbine (Rauwolscine) From Rauwolfia Species”—Pawan Kumar Goel.

This above invention discloses a method for the extraction of alpha yohimbine from the roots, stem and leaves of Rauwolfia species and determination of the purity of the compound using HPLC. The above invention also belongs to the same applicant as of present invention. It is the closest Prior art.

The process of this prior art patent uses a precipitation method including alternate steps of acidification and alkalization along with organic solvents. Aromatic non-polar solvent toluene was used for the process of extraction by maintaining 40-50° C. Temperature and 9 to 9.5 pH by addition of alkali followed by the extraction using an acid preferably Tartaric acid. Extraction is followed by alkalization and extraction with chloroform for the purification of the product. Methyl alcohol was used for the precipitation of the extracted product. Other organic acids such as tartaric acid, oxalic acid were used for lowering of the pH at various steps of acidification.

The present invention is by same applicant as with improvements, which have resulted in a new process, which is faster, more economical and results in much higher yield of about 50-65% more as compared to prior art process.

The present invention or process is using Alcoholic solvent “Methanol” instead of using Aromatic solvent toluene at normal pH range and 40-55° C. temperature which is basically enhancing the yield of the final extract (50-65%) increase in yield). The above invention involves multiple acidification and alkalization steps for extraction as well as purification of the final product which are complex and require more man hours, whereas in the present invention, the number of steps are less as after organic solvent alkali extraction, the present invention involves the extraction using an organic solvent that is Chloroform without the use of any acid. The process of acidification is not involved in the present invention, which reduces handling losses of chemicals, making process more economical than existing process. In already existing process, organic acids such as tartaric acid, oxalic acid, etc. were used for lowering pH whereas in the present process, these are not used at all. The present invention also eliminates the use of ethyl acetate during purification. Higher yield i.e. about 50-65% increase is obtained using the new process as compared to the already existing process, which has considerable industrial importance.

### Prior Art vs Present Invention

| Prior Art Process | | Present invention |
|---|---|---|
| Pat. No. U.S. 10,730,869 B2 “Novel Process | | “Improved industrial process for the extraction |
| for Extracting Alpha Yohimbine (Rauwolscine) | | of Alpha yohimbine from Rauwolfia species”- |
| From Rauwolfia Species”- Pawan Kumar Goel | | Pawan Kumar Goel |
| 1. | Aromatic solvent toluene (NON | Alcoholic solvent Methanol (Polar solvent) is |
| | POLAR) was used for extraction | used for extraction. (Temp: 40-55° C.). Being |
| | (Temp: 40-50° C.) | highly polar in nature, Methanol helps in |
| | | extraction of all the alkaloids and since a few |
| | | steps have been omitted, the losses at these |
| | | steps have been minimized and further use of |
| | | methanol helps in the extraction of product |
| | | with higher yield. |
| 2. | More no. of steps including repeated | Lesser no. of steps are involved in extraction of |
| | alkalization (takes 4 hours each) and | product because extraction is carried out using |
| | Acidification (takes 4 hours each) | Methanol. Being highly Polar in nature, |
| | | methanol helps in extraction of all the alkaloids |
| | | without basic treatment. |
| 3. | Lower yield | Higher Yield (about 50%-65% more yield) |
| | 400 mg/100 g dry leaves | 600 mg-670 mg/100 g dry leaves. |
| 4. | Raw materials such as organic acids | Only hydrochloric acid is used for the process |
| | including Tartaric acid and oxalic acid | of acidification and other raw materials such as |
| | and hexane, Ethyl acetate were used in | oxalic acid, tartaric acid etc are not used in the |
| | this method. | present invention, which helps in reducing |
| | | losses. Tartaric acid is not used because no |
| | | basification is being done at extraction stage. |
| | | Use of hydrochloric acid easily precipitates out |
| | | all required alkaloids, since it is a strong acid. |
| | | The ethyl acetate step which is used to take |
| | | approximately 24 hours has been eliminated. |

Patent Application No: US20120184576A1 Discloses “Antipsychotic Agents and Standardized Antipsychotic Fractions from Rauwolfia tetraphylla and Process of their Isolation”

The above invention discloses a process for obtaining antipsychotic canescens extracts and alkaloids including alpha yohimbine from the leaves of Rauwolfia tetraphylla. The above invention uses organic solvent alkali and acidic extraction steps for the extraction of the compound and uses column chromatography for the separation of the alpha yohimbine from the other compound or simply for the purification of the alpha yohimbine.

The present invention differs from the above invention, as in the above invention, separate technique was used for the extraction that is organic solvent acid and alkali extraction and separate technique was used for the purification of the compound that is column chromatography whereas in present invention, the compound is being purified using acid base extraction technique without the use of column chromatography. The present invention results in high yield of the compound as compared to the already existing invention. Also the present invention involves the extraction of alpha yohimbine only for the commercial use whereas the above invention involves the extraction of other alkaloids also along with alpha yohimbine.
Patent Application No: CN102863439A Discloses “Method for extracting yohimbine hydrochloride from yohimbe barks”

The above invention discloses a method for the production of yohimbine hydrochloride from the yohimbe (Pausinystalia yohimbe) barks. The method involves the use of organic solvents and various acids for the extraction. Vacuum drying and resin column methods were used for the drying and concentration of the product.

The present invention involves the extraction of Alpha-yohimbine from a completely different species that is Rauwolfia tetraphylla hence, is completely different from the above invention and also the present invention does not involve the use of critical and time step methods such as resin column chromatography, hence is simple and economically feasible.

### JOURNALS/BOOKS/LITERATURE

Arvind Kumar, M. K. Bhardwaj, Upadhyay A. K., Anupa Tiwari and Bikram Deo Ohdar. Quantitative determination of Yohimbine alkaloid in the different part of the Rauwolfia tetraphylla. Journal of Chemical and Pharmaceutical Research. J. Chem. Pharm. Res., 2011, 0.3(2): Pages 907-910.

The above publication discloses a method for extraction and quantitative determination of yohimbine from different parts of Rauwolfia species. In above publication, the roots, stems and leaves were used and extraction was carried out using organic solvent methanol and by centrifugation and the solvent was further removed using filtration only. Quantitative determination was conducted using HPTLC and it was observed that 6.11% of yohimbine was present in leaves of Rauwolfia tetraphylla and in stems and roots, it was absent.

The present invention discloses a method for the extraction of alpha yohimbine using organic solvent acidic and basic extraction method and acid-base purification method from Rauwolfia species. The present invention produces high yield and high purity of alpha yohimbine. The present invention differs from the above invention as in above invention the extraction was carried out using organic solvent only whereas in the present invention, precipitation method along with use organic solvents, acid and base was used for extraction with alternate washing steps using halogenated solvent (Chloroform). Also the present invention is cost effective as it does not require the use of certain sophisticated equipments such as column.
Ram Kishore Verma, Shikha Gupta, Madan Mohan Gupta, Santosh Kumar Srivastava. A Simple Isocratic HPLC Method for the Simultaneous Determination of Antipsychotic Indole Alkaloids in Rauwolfia tetraphylla. Journal of Liquid Chromatography and Related Technologies, 2012.

The above publication discloses a simple isocratic HPLC method for quantification of α-yohimbine, isoreserpiline and tetrahydroalstonine. Samples were analysed by reverse phase chromatography on a Waters spherisorb ODS2 column using isocratic elution with acetonitrile containing 0.1% TEA and water containing 0.1% TFA (35:65, v/v) at a flow rate of 1 mL/min, a column temperature of 30 C and UV detection at 210 nm.

The present invention differs from the above publication as in the above invention the analyses of samples were done using certain complex and sophisticated methods and equipment such as reverse phase chromatography and Detection of extracted compounds was carried out using UV detection technique. Also, the above process is complicated as it uses a lot of solvents whereas the present invention uses only few solvents and hence is simple, convenient, commercially economic and less time-consuming process and is producing the product with high yield and purity.
Sarika R. Deshmukh, Dhanashree S. Ashrit and Bhausaheb A. Patil. Extraction and Evaluation of Indole Alkaloids from Rauwolfia serpentina for their Antimicrobial and Antiproliferative Activities. International Journal of Pharmacy and Pharmaceutical Sciences. Vol 4, Suppl 5, 2012.

The above study deals with the extraction and evaluation of Indole alkaloids from Rauwolfia serpentina. In the above process, Indole alkaloids were extracted using ethanol at 25° C. for about 12 hours. Extraction is followed by phytochemical screening, which was conducted using various tests, including Molisch's test, Fehling's test. Tannins test etc. And qualitative assessment was carried out using TLC and HPLC methods. Also anti-proliferative and antimicrobial activity of extract of Rauwolfia serpentine was detected. 11.27% crude extract was obtained in the above process.

The present invention discloses the extraction of alpha yohimbine from Rauwolfia species using organic solvent methanol and alternative acidification and alkalization steps along with precipitation of the extracted compound hence is completely different from the above invention. Also, the present invention is simple and not using any complex chromatography technique and chemicals, hence is cost effective and also producing quite high yield of alpha yohimbine as compared to the already existing processes.
Sunil Kumar, Awantika Singh, Vikas Bajpai, Mukesh Srivastava, Bhim Pratap Singh, Sanjeev Ojhae and Brijesh Kumara. Simultaneous Determination of Bioactive Monoterpene Indole Alkaloids in Ethanolic Extract of Seven Rauvolfia Species using UHPLC with Hybrid Triple Quadrupole Linear Ion Trap Mass Spectrometry. July 2016. Phytochemical Analysis.

The above publication discloses, a validated, rapid, sensitive and selective ultra HPLC coupled with hybrid triple quadrapole linear ion trap mass spectroscopy method in multiple reaction monitoring mode for simultaneous determination of bioactive MIAs in ethanolic extract of seven Rauwolfia species. The powdered plant materials in above process were extracted using ethanol by 30 minutes sonication using an ultra-sonic water bath. HPLC combined with hybrid triple quadrapole linear on trap mass spectroscopy technique was used for the analysis of the extracted alkaloids from seven Rauwolfia species.

The present invention differs from the already existing process because present invention does not use complex methodology and techniques such as sonication and chromatography for extraction and analysis of extract and also the extraction in present invention was conducted using organic solvent methanol along with use of acid base and other precipitation agents and halogenated solvents such as chloroform.

### Commercial Information

### Alpha Yohimbine

https://www.amazon.in/Team-Six-Supplements-Alpha-Yohimbine/dp/801LXDJ207

The above commercialized product was extracted from the bark of Rauwolfia vomitoria and is used as a dietary supplement, whereas the product disclosed in above invention as extracted from root, stem and leaves of Rauwolfia tetraphylla.

### OBJECTS OF THE PRESENT INVENTION

The main object of the present invention is to disclose an improved process for the extraction of alpha yohimbine from Rauwolfia species resulting in higher yield.

One more object of the present invention is to disclose simple and economically feasible industrial process for the extraction of alpha yohimbine from Rauwolfia species.

Still another object is to disclose that the extract obtained by the process of present invention contains yohimbine hydrochloride as a marker compound in analytically detectable amounts of more than 0.1% of the extract.

### SUMMARY OF THE INVENTION

The present invention discloses an improved process for the extraction of alpha yohimbine from leaves of Rauwolfia species, preferably Rauwolfia canescens/Rauwolfia tetraphylla. The process of present invention gives, about 50-65% increased yield of alpha yohimbine i.e. 600-670 mg/100 g dry leaves as compared to prior art processes. Further, the extract obtained by the process of present invention contains yohimbine hydrochloride as a marker compound in analytically detectable amounts of more than 0.1% of the extract.

The extraction process is simple, convenient and commercially economical as it does not involve the use of sophisticated methods and techniques such as chromatography and mass spectrometry. Also, the disclosed process uses lesser number of solvents, which helps in the elimination of handling losses being suffered in the old processes. The disclosed process involves specific extraction steps such as organic solvent extraction, acidification, alkalization etc. The disclosed process involves the use of polar alcoholic solvent “Methanol” instead of non-polar aromatic solvent “Toluene” which results in production of extract with higher yield. Moreover, Carbon treatment is being used to remove color from the sample.

### BRIEF DESCRIPTION OF THE DRAWINGS

FIG. 1: HPLC Analysis for sample: Blank

FIG. 2: HPLC Analysis Sample: 001

FIG. 3: HPLC Analysis Sample: 002

FIG. 4: HPLC Analysis Summary (Compound) Detector A

FIG. 5: HPLC Analysis to determine Purity (Lot No: 01)

FIG. 6: HPLC Analysis Sample ID: CR/YOHAL/19-20

FIG. 7: HPLC Analysis to determine Purity (Lot No: 02)

FIG. 8: HPLC fingerprint of extract obtained by prior art process vs new process

FIG. 8 A: HPLC fingerprint of extract of reference standard showing yohimbine hydrochloride content

FIG. 8 B: HPLC fingerprint of extract by new process of present invention showing yohimbine hydrochloride as marker compound with content of more than 0.1% of extract.

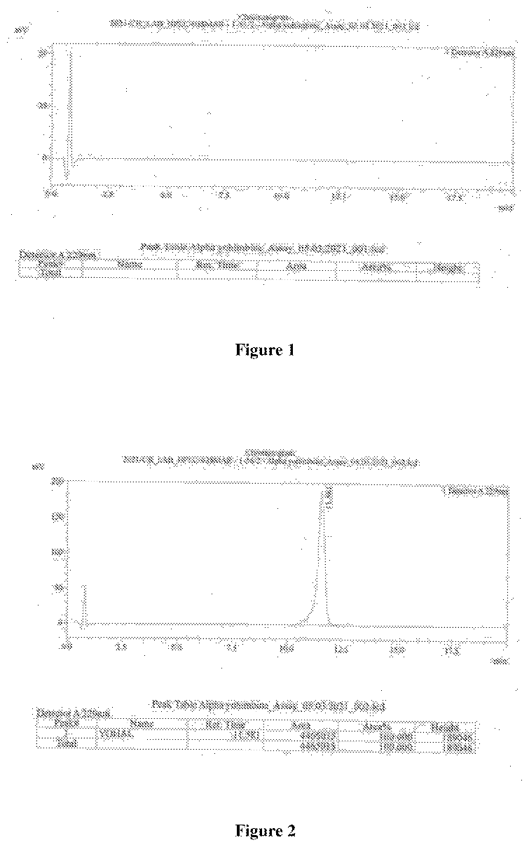

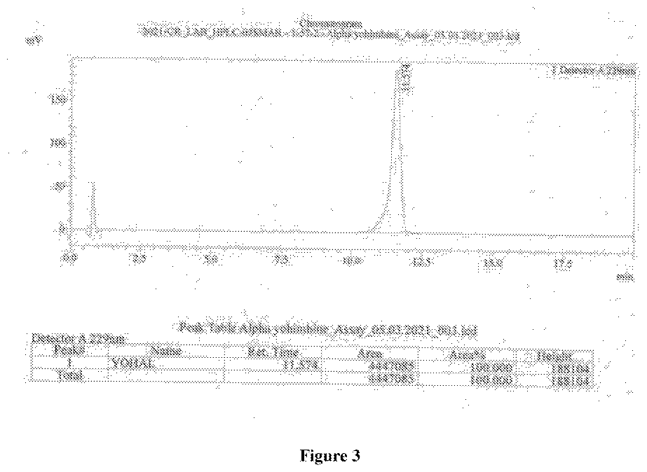

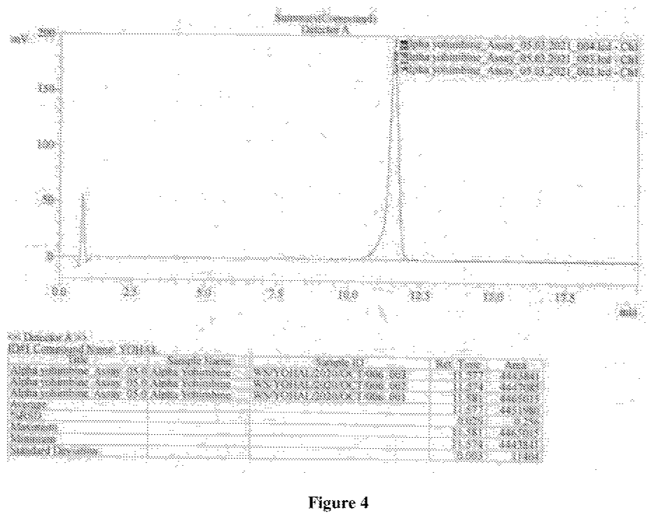

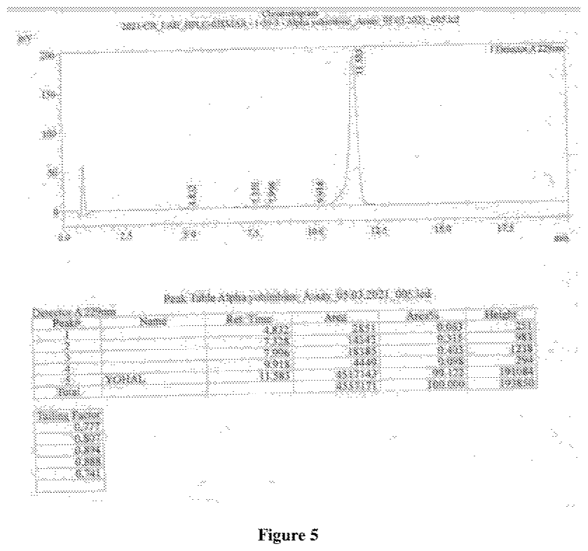

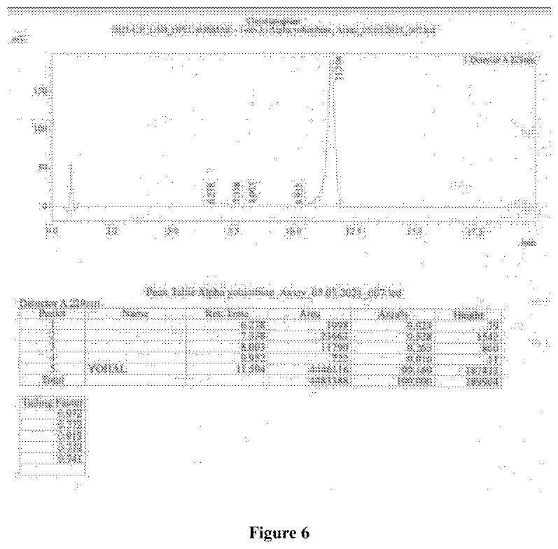

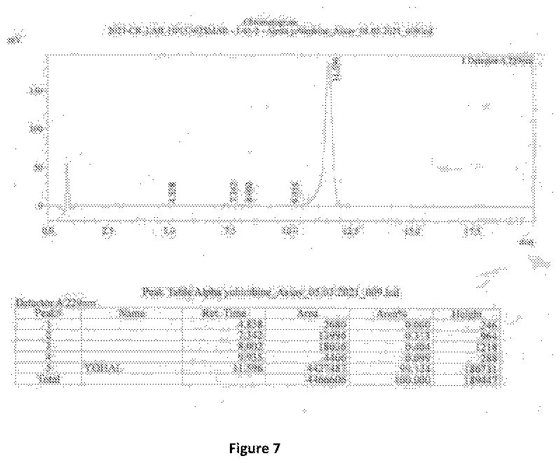

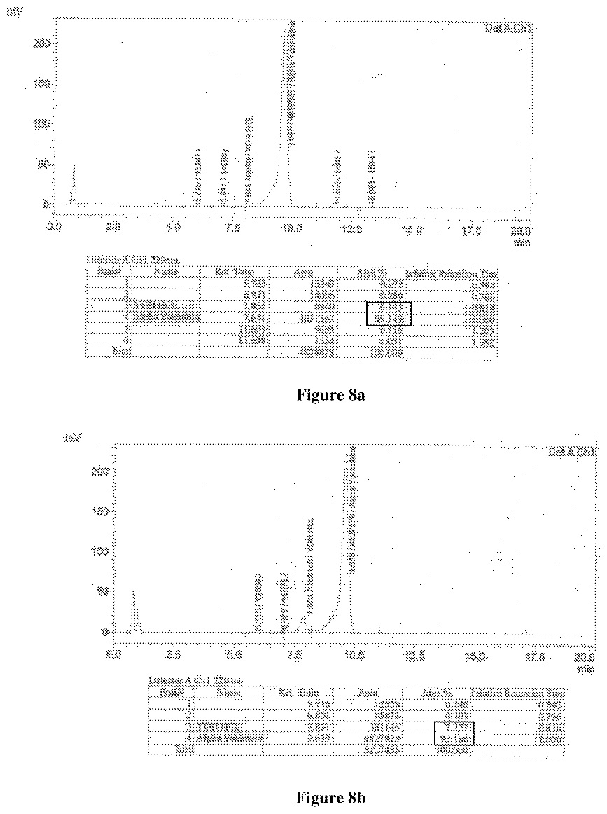

### DETAILED DESCRIPTION OF THE INVENTION

The following description is of best-contemplated mode of carrying out the invention. The description is made for the purpose of illustrating the general principles of the invention and should not be taken in a limiting sense.

The present invention discloses an improved commercially viable industrial process for the extraction of alpha yohimbine form the leaves of Rauwolfia canescens/Rauwolfia tetraphylla. The process disclosed in present invention produces remarkable higher yield of the final material (About 50-65%) increased yield) i.e. 600-670 mg/100 g of dry leaves (Yield in prior art process was 400 mg/100 g of dry leaves. Also, the improved process is commercially economical along with quite simple methodology.

Extraction is the first step to separate the desired natural constituents from the parts of plants. Various methods of natural product extraction are available including, organic solvent extraction, distillation, pressing, sublimation, etc. out of these methods, the simplest and most widely used technique for natural product extraction is “Solvent Extraction” technique. The basic principle associated for extraction with solvent extraction process is that, the solvent penetrates into the solid matrix which dissolves the desired solute depending upon the polarity of the solvent based on the law of similarity and known fact of chemistry that ‘Like dissolves like’ that is the polar solvents will dissolve the polar compounds and non-polar solvents will dissolve non polar compounds. Hence solvents are usually selected on the basis of compound of interest to be extracted.

For the extraction of polar compounds polar solvents are being used whereas non polar or slightly polar compounds are extracted with the help of non-polar solvents. Once, the solvent will dissolve the solute, the solute will diffuse out of the solid matrix, further the extracted solutes are collected and purified. The purification is usually carried out to obtain high purity compound. The extraction process can be coupled with High Performance Liquid chromatography in order to obtain highly pure product along with high yield of the desired product.

Other than polarity, particle size, pH and Temperature are the other factors that affect the overall process of extraction. Smaller or finer the particle size, better will be the extraction. Usually, high temperatures increase the solubility and diffusion of the solute, but if a specific range of temperature exceeds, it results in the loss of the solvent which as a result produces the extract with no. of undesirable impurities. pH that is acidic or basic nature of solvent can also affect the extraction process as the addition of acid or base to organic solvent can results in the separation of desired secondary metabolites.
The present invention follows the technique of organic solvent extraction along with alternate steps of acidification and alkalization in order to obtain extraction of alpha yohimbine with higher yield.

### METHODOLOGY

The extraction procedure involves various steps. Some of the solvents can be changed without departing from the scope of the invention. Three different approaches can be used which are illustrated as Examples 1 and 2 below:

### Example-1

Process involves following steps:
Step 1: Sample Collection—The leaves (As leaves contain highest quantity of alpha yohimbine) of Rauwolfia species are collected from Southern Part of India mainly Madhya Pradesh. Chhattisgarh, Tamil Nadu, Kerala and other parts of India. These leaves are dried and grounded/Powdered in order to reduce the size of the parts for the process of extraction.
Step 2: Organic Solvent Extraction—Alcoholic solvent “Methanol” is being used for the extraction of desired compound. The compound is extracted multiple times usually 3-4 times by using 3-4 volumes of alcoholic solvent “Methanol” with respect to the weight of leaves each time. The extraction process is carried out by maintaining a temperature of about 40-55° C. each time. After extraction, the solvent is reduced off or removed by the process of distillation and the remaining product is mixed with purified or demineralized water.
Step 3: Halogenated solvent washing—The solution is then washed with the help of Halogenated solvent such as Chloroform or Dichloromethane. Washing is being repeated 3-4 times by taking solvent (Halogenated) in ratio of 1:1 (one part of mixture from step 3 and one part of solvent).
Step 4: Alkalization of Aqueous layer—Washing with halogenated solvent results in the separation of desired product and other impurities. The desired compound will remain in the aqueous layer. Hence, the aqueous Layer is basified/alkalized with the help of 20-25% alkali solution that is 20-25% Ammonium hydroxide which will result in rise of pH to about 9-9.5 pH. After Alkalization, the extract is again washed 3-4 times using 1:1 ratio of halogenated solvent Chloroform/Dichloromethane. The solvent is then reduced off or removed by distilling it off.
Step 5: Precipitation step—The filtrate/extract is co-distillated with alcoholic solvent “Methanol” at a volume approximately 1/15 to 1/10 times the weight of dry herb. The extract is then acidified (pH 2-2.5) using Hydrochloric acid at 40-55° C. The temperature that is 40-55° C. and pH 2-2.5 is maintained for about 2-3 hours for the process of precipitation. After 2-3 hours the extract is filtered thoroughly.
Step 6: Purification—The purification or removal of undesired impurities form the alpha yohimbine extract is obtained by dissolving the recovered precipitate in demineralized water and addition of alkali in order to increase the pH from 2-2.5 to 9-9.5. This pH is maintained for about 2-3 hours at 40-55° C. temperature. After alkalization, the extract is filtered and dried. Further the precipitate is dissolved in alcoholic solvent “Methanol”. The solution is further given carbon treatment in order to remove colored impurities form the filtrate/extract.
Step 7: Final Purification—The pH of the solution is again lowered with the help of hydrochloric acid and maintained the pH 2-2.5 for about 2-3 hours at 40-55° C. Finally, the solution is filtered in order to separate the precipitate from the solution. The solution is discarded and final precipitate which is of yellowish white to off white in color is dried and stored at ambient temperature or room temperature 20-30° C.

### Example 2

Step 1: Organic Solvent Extraction—Alcoholic solvent Methanol is being used for the extraction of desired compound. The compound is extracted multiple times usually 3-4 times by using 3-4 volumes of alcoholic solvent with respect to the weight of leaves each time. The extraction process is conducted by maintaining a temperature of about 40-55° C. each time. After extraction, the solvent is reduced off by the process of distillation and the remaining product is mixed with purified or demineralized water.
Step 2: Hexane (Petroleum Ether) washing—The solution/extract is further washed with Hexane or petroleum ether in order to remove the oily impurities, hence helps in recovery of high purity extract.
Step 3: Halogenated solvent Washing—Washing is followed by multiple time preferably 3-4 times washing with 1:1 (one part of extract and one part of solvent) ratio of halogenated solvent i.e., Chloroform or Dichloromethane.
Step 4: Alkalization of Aqueous layer—Washing with halogenated solvent results in the separation of desired product and other impurities. The desired compound will remain in the aqueous organic layer. Hence, the aqueous Layer is basified/alkalized with the help of 20-25% alkali solution that is 20-25% Ammonium hydroxide which will result in rise of pH to about 9-9.5 pH. After Alkalization, the extract is again washed 3-4 times using 1:1 ratio of halogenated solvent Chloroform. The solvent is then reduced by distilling it off.
Step 5: Precipitation step—The filtrate/extract is co distillated with alcoholic solvent “Methanol” at a volume approximately 1/15 to 1/10 times the weight of dry herb. The extract is then acidified (pH 2-2.5) using Hydrochloric acid at 40-55° C. The temperature that is 40-55° C. and pH 2-2.5 is maintained for about 2-3 hours for the process of precipitation. After 2-3 hours the extract is filtered thoroughly.
Step 6: Purification—The purification or removal of undesired impurities form the alpha yohimbine extract is obtained by dissolving the recovered precipitate in demineralized water and addition of alkali in order to increase the pH from 2-2.5 to 9-9.5. This pH is maintained for about 2-3 hours at 40-55° C. temperature. After alkalization, the extract is filtered and dried. Further the precipitate is dissolved in alcoholic solvent. The solution is further given carbon treatment in order to remove colored impurities form the solution.
Step 7: Final Purification—The pH of the solution is again lowered with the help of hydrochloric acid and maintained the pH 2-2.5 for about 2-3 hours at 40-55′C. Finally, the solution is filtered in order to separate the precipitate from the solution. The solution is discarded and final precipitate which is of yellowish white to off white in color is dried and stored at ambient temperature or room temperature 20-30° C.

TABLE 1
Comparison of HPLC Chromatograms of Alpha Yohimbine

| | | | | | Batch No.: | |
|---|---|---|---|---|---|---|
| | | | | | CR/YOHAL/ | |
| | Lot No.: 01 | | Lot No.: 02 | | 19-20/DEC/03 | |
| | Retention | | Retention | | Retention | |
| S. No. | Time | % Area | Time | % Area | Time | % Area |
| 1 | 4.832 | 0.063 | 4. [symbol] | 0.060 | — | — |
| 2 | — | — | — | — | 6.278 | 0.024 |
| 3 | 7.328 | 0.315 | 7.342 | 0.313 | 7. [symbol] | 0. [symbol] |
| 4 | 7.096 | 0.403 | 8.002 | 0.404 | 8.003 | 0.263 |
| 5 | 9.918 | 0.098 | 9. [symbol] | 0.099 | 9.952 | 0.016 |
| 6 | 11.583 | 99.122 | 11.596 | 99.124 | 11.594 | 99.169 |

| | | | | | |
|---|---|---|---|---|---|
| Chromotographic Purity | 99.122% | — | 99.124% | — | 99.169% |
| Total Impurities | 0.878% | — | 6.876% | — | 0.831% |
| Assay (By HPLC) | 90.6% | — | 90.2% | — | 90.3% |

[symbol]

indicates data missing or illegible when filed

It is clear from the above table that purity of product obtained by present process (Lot 01 and 02) is almost the same as that of purity of product prepared by earlier prior art process of the applicant himself. However, the present process is economical, faster and results in higher yield of 50-65% than earlier process. Additionally, the extract obtained by the process of present invention contains yohimbine hydrochloride as a marker compound in analytically detectable amounts of more than 0.1% of the extract.

### Novelty, Inventive Step and Industrial Application

### Novelty

The present invention discloses an improved process for the extraction of Alpha yohimbine from Rauwolfia species i.e. Rauwolfia canescens/Rauwolfia tetraphylla. The improved process results in production of compound with 50-65% more yield as compared to already existing processes. The average yield from already existing process was 400 mg/100 g dry leaves (4 Kg/Ton dry leaves) whereas the yield from proposed process has varied from 6.0 to 6.7 Kg/Ton of dry leaves of plant Rauwolfia species which was not obtained in any other processes. The Novelty lies in the use of methanol (polar molecule) instead of tolune (non-polar molecule) and also acid-base steps for the extraction of desired compound. Also, use of lesser number of steps and lesser number of solvents/reagents makes the improved process more economical than prior art processes.

### Inventive Step

The inventive step of the present invention lies in the disclosure of method which produces quite high yield of alpha yohimbine—about 50-65% increased yield as compared to other methods, used for the extraction process. Inventive step in the terms of technical advancement over prior art, lies in use of methanol (polar molecule) instead of toluene (non-polar), elimination of use of organic acids and acidification and alkalization steps to achieve high yield and purity of desired extract. Methanol being a highly polar solvent, extracts all the alkaloids and since a few steps have been omitted, the losses at these steps have been minimized, thus increasing the yield at final stage. The disclosed process is commercially economical as lesser number of organic solvents and reagents are being used.

### INDUSTRIAL APPLICATION

Alpha yohimbine is of great medicinal importance. It is used to treat erectile dysfunction and promote weight loss. It is also used to boost mood, to improve athletics' performance, to treat Angina, diabetic neuropathy etc. Moreover, the process used in present invention for extraction of alpha yohimbine is simple and commercially economical. Hence the present invention is of great industrial importance.

I claim:

1. An improved industrial process for the extraction of Alpha yohimbine from Rauwolfia species preferably Rauwolfia canescens and Rauwolfia tetraphylla, consisting of following steps:
i) Powdering the dried leaves of plant Rauwolfia species;
ii) Extracting the compound using 1:3 to 1:4 wt./vol. of Methanol with respect to the weight of leaves at a temperature of about 40-55° C. each time;
iii) Repeating step ii) for 3.4 times;
iv) Distilling off the solvent from extract, so as to prepare a concentrated extract which is mixed with purified or demineralized water;
v) Washing the solution obtained in step (iv) with the help of halogenated solvent such as Chloroform or Dichloromethane in a ratio of 1:1 (one part of solid and one part of solvent);
vi) Repeating washing as in Step v 3-4 times to obtain an aqueous layer containing the desired compound which is alkalized with the help of 20-25% Alkali solution (Ammonium hydroxide solution) to a pH of about 9-9.5;
vii) After Alkalization, washing the extract 3-4 times using halogenated solvent Chloroform/Dichloromethane in a ratio of 1:1;
viii) Removing or reducing the solvent by distilling it off; by distilling it off;
ix) Co-distilling the filtrate/extract obtained in step VIII with alcoholic solvent Methanol at a volume approximately 1/15 to 1/10 times the weight of dry herb;
x) Acidifying the reduced extract (pH 2.2.5) using Hydrochloric acid at 40-55° C. maintaining a pH of 2-2.5 for about 2-3 hours followed by filtration thoroughly;
xi) Dissolving the recovered precipitate in demineralized water and alkalizing it in order to increase the pH from 2-2.5 to 9-9.5 for about 2-3 hours at 40-55° C. temperature;
xii) After alkalization, filtering, drying and precipitating the extract and then dissolving it in alcoholic solvent e.g. methanol in a ratio of 1:10 to 1:15 vol/vol;
xiii) Treating the solution with carbon to remove colored impurities from the filtrate/extract;
xiv) Lowering pH of the solution with the help of hydrochloric acid to 2-2.5 and stirring for about 2-3 hours at 40-55° C.;
xv) Drying the final precipitate of alpha yohimbine which is yellowish white to off white in color and storing at ambient temperature or room temperature 20-30° C.

2. The improved process as claimed in claim 1 wherein the yield of alpha yohimbine is 6.0 to 6.7 Kg/Ton of dry leaves of plant Rauwolfia species.

3. The improved industrial process as claimed in claim 1 wherein an additional step of washing the solution/extract obtained in step (iv) with Hexane or petroleum ether in order to remove the oily impurities, is carried out optionally.

4. The extract obtained by the process as claimed in claim 1 wherein the same contains yohimbine hydrochloride as a marker compound in analytically detectable amounts of more than 0.1% of the extract.
